(19) United States
(12) Patent Application Publication
(10) Pub. No.: US 2010/0249238 A1
(43) Pub. Date: Sep. 30, 2010

(54) PHARMACEUTICAL COMPOSITIONS COMPRISING SEMICARBAZONES AND THIOSEMICARBAZONES AND METHOD FOR TREATING INFLAMMATORY, PAINFUL AND FEBRILE CONDITIONS AND PREVENTING SIGNS AND SYMPTOMS OF INFLAMMATION

(76) Inventors: Ruben Dario Sinisterra, Ouro Preto (BR); Marcio De Matos Coelho, Belo Horizonte (BR); Heloisa De Oliveira Beraldo, Belo Horizonte (BR); Maria Carolina Doretto, Vespasiano (BR); Rafael Pinto Vieira, Belo Horizonte (BR); Leticia Regina De Souza Teixeira, Belo Horizonte (BR); Silvia Passos Andrade, Belo Horizonte (BR)

Correspondence Address: NIXON & VANDERHYE, PC, 901 NORTH GLEBE ROAD, 11TH FLOOR, ARLINGTON, VA, 22203, US

(21) Appl. No.: 12/667,351

(22) PCT Filed: Jan. 25, 2008
(86) PCT No.: PCT/BR2008/000023

§ 371 Date: May 13, 2010

(30) Foreign Application Priority Data
Jul. 2, 2007 (BR) C10200751-7

(51) Int. Cl. A61K 31/175; A61P 29/00; C07C 281/14

(52) U.S. Cl. 514/590; 564/36

(57) ABSTRACT

The present patent application refers to pharmaceutical compositions comprising at least a semicarbazone or a thiosemicarbazone, or a pharmaceutically acceptable salt, hydrated or solvated thereof, for the treatment of inflammatory, febrile and painful inflammatory conditions, inflammatory edema and peripheral or central neurophatic painful conditions or prevention of signs and symptoms of inflammation. Claim also pharmaceutical compositions comprising at least a semicarbazone, or a thiosemicarbazone, or a pharmaceutically acceptable salt, hydrated or solvated thereof and a therapeutically effective amount of these compounds, mixed or included in a pharmaceutically acceptable carrier or excipient or a thiosemicarbazone is provided as sustained or controlled release systems for human and veterinary use in solutions or in the solid state.

### FIELD OF INVENTION

The present patent application claims pharmaceutical compositions comprising at least a semicarbazone or a thiosemicarbazone, or their pharmaceutically acceptable salts, hydrated or solvated thereof, for the treatment of inflammatory, febrile and painful inflammatory conditions, inflammatory edema and peripheral or central neurophatic painful conditions or prevention of signs and symptoms of inflammation. It also claims pharmaceutical compositions comprising at least a semicarbazone, or a thiosemicarbazone, or their pharmaceutically acceptable salts, hydrated or solvated thereof and a therapeutically effective amount of these compounds, mixed or included in a pharmaceutically acceptable carrier or excipient, or controlled release systems for human and veterinary use in solutions or in the solid state. As examples of pharmaceutically acceptable excipients for solid formulations to be used for oral administration, starch, lactose, microcrystalline cellulose, hydroxypropyl methylcellulose, talc and magnesium stearate or mixtures thereof can be cited. As examples of pharmaceutically acceptable excipients for liquid formulations to be used for oral administration, glycol propylene, glycerol, sorbitol, saccharose, glucose and fructose can be chosen thereof. As examples of pharmaceutically acceptable excipients for liquid formulations to be used for parenteral administration, polyvinyl pyrrolidone, cremophor, tween 80 can be chosen thereof.

### BACKGROUND OF INVENTION

Thiosemicarbazones of formula

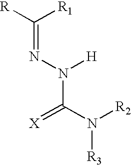

are compounds with a large range of biological applications, presenting antitumoral, antiviral, antibacterial, antimalarial, antituberculosis, antifungal, anti-HIV and anticonvulsant activities [Beraldo, H.; Gambino, D.; Minireviews in Medicinal Chemistry, 4, 159, 2004, West, D. X.; Padhyé, S. B.; Sonawane, P. B., Structure and Bonding, 76, 1, 1991; Dimmock, J. R., Pandeya, S. N., Quail, J. W., Pugazhenthi, U., Allen, T. M., Kao, G. Y., Balzarini, J., DeClercp, E., Eur. J. Med. Chem., 30, 303, 1995].

Semicarbazones are analogues to the above mentioned compounds in which oxygen replaces sulfur. Many studies have reported the anticonvulsant activity of semicarbazones [Beraldo, H.; Gambino, D.; Minireviews in Medicinal Chemistry, 4, 159-165, 2004; Dimmock, J. R., Pandeya, S. N., Quail, J. W., Pugazhenthi, U., Allen, T. M., Kao, G. Y., Balzarini, J., DeClercq, E., Eur. J. Med. Chem., 30, 303, 1995; Dimmock, J. R.; Sidhu, K. K.; Thayer, R. S.; Mack, P.; Duffy, M. J.; Reid, R. S.; Quail, J. W.; Pugazhenthi, U.; Ong, A.; Bikker, J. A.; Weaver, D. F., J. Med. Chem., 36, 16, 1993; Dimmock, J. R.; Puthucode, R. N.; Smith, J. M.; Heltherington, M.; Quail, W. J.; Pughazenti, U.; Leshler, T.; Stables, J. P., J. Med. Chem., 39, 3984, 1996]. In particular, compounds derived from arylsemicarbazones present anticonvulsant activity [Kadaba, P. K.; Lin, Z.; U.S. Pat. No. 5,942,527, 1999; Dimmock, J. R.; Puthucode, R. N.; WO9640628, MX9709311, JP11506109, U.S. Pat. No. 5,741,818, 1997; Fujibayashi, Y.; Yokoyama, A.; U.S. Pat. No. 5,843,400, 1996].

Structural variations leading to significant modifications of the biological activity of semicarbazones and thiosemicarbazones have been reported [West, D. X.; Padhyé, S. B.; Sonawane, P. B., Structure and Bonding, 76, 1, 1991; Kadaba, P. K.; Lin, Z.; U.S. Pat. No. 5,942,527, 1999].

In the state-of-the-art, it is reported that semicarbazones and thiosemicarbazones present anticonvulsant activity in two experimental models of epilepsy: the pentylenetetrazole (PTZ) and the maximum electroshock (MES) screen [Dimmock, J. R.; Sidhu, K. K.; Thayer, R. S.; Mack, P.; Duffy, M. J.; Reid, R. S.; Quail, J. W.; Pugazhenthi, U.; Ong, A.; Bikker, J. A.; Weaver, D. F., J. Med. Chem., 36, 16, 1993; Dimmock, J. R.; Pandeya, S. N.; Quail, J. W.; Pugazhenthi, U.; Allen, T. M.; Kao, G. Y.; Balzarini, J.; DeClercq, E., Eur. J. Med. Chem., 30, 303, 1995; Dimmock, J. R.; Sidhu, K. K.; Tumber, S. D.; Basran, S. K.; Chen, M.; Quail, J. W.; Yang, J.; Rozas, I.; Weaver, D. F., Eur. J. Med. Chem., 30, 287, 1995; Dimmock, J. R.; Puthucode, R. N.; Smith, J. M.; Heltherington, M.; Quail, W. J.; Pughazenti, U.; Leshler, T.; Stables, J. P., J. Med. Chem., 39, 3984, 1996; Dimmock, J. R.; Vashishtha, S. C.; Stables, J. P., Eur. J. Med. Chem., 35, 241, 2000; Kadaba, P. K.; Lin, Z., U.S. Pat. No. 5,942,527, 1999; Dimmock, Puthucode, R. N., WO9640628, MX9709311, JP11506109, U.S. Pat. No. 5,741,818, 1997; Fujibayashi, Y.; Yokoyama, A., U.S. Pat. No. 5,843,400, 1996].

The patents that report the anticonvulsant activity of semicarbazones and thiosemicarbazones are described below.

U.S. Pat. No. 5,942,527 (1999)—Kadaba et al. prepared new pharmaceutical compositions containing hydrazones, hydrazines, thiosemicarbazones and semicarbazones and evaluated their anticonvulsant activity in the model of electroshock-induced seizures in rats. The compositions were active after oral administrations in doses of 100 mg/Kg and presented low neurotoxicity.

U.S. Pat. No. 5,741,818 (1997), (MX9709311, WO9640628, AU9659938, FI9704447, N09705663, EP836591, CZ9703874, NZ309707, HU9802637, JP11506109, BR9609408, AU715897, KR99022408)—Dimmock et al. prepared semicarbazones derived from 4-phenoxy- or 4-phenylthio-benzaldehyde and evaluated their anticonvulsant activity in the model of electroshock induced-seizures in rats. The compounds presented no neurotoxicity in doses up to 500 mg/Kg.

WO9406758 (1996)—Dimmock prepared aryl semicarbazones and evaluated their anticonvulsant activity. These compounds were more active than phenyloin, phenobarbital and the corresponding semicarbazides. They are stable, can be administered orally and present low or no neurotoxicity.

WO2003066038 (2003) Beraldo et al. report a process to prepare semicarbazones and/or thiosemicarbazones formulations using cyclodextrins and their derivatives and products obtained by this process. The formulations allowed 65-85% anticonvulsant dose reduction. A pain killer effect has been observed for the studied compounds.

### Pathophysiology of Pain and Inflammation

Infection, chemical and physical stimuli, hypoxia, autoimmune reactions, among other endogenous and exogenous factors, may cause cell lesion and, according to the magnitude and duration, may also cause cell death. The presence of these noxious factors induces a local and non-specific response, usually with a protective function, denominated inflammation. This response contributes to eliminate the stimulus that induced the cell lesion and also the necrotic tissue that resulted from this lesion, allowing tissue regeneration [Tracey K. J., Nature, 420, 853, 2002].

One of the symptoms associated with the inflammatory response and also with some pathologic conditions not associated with inflammation that represents the most important cause of suffering for the patients is pain. According to the International Association for the Study of Pain (IASP), pain is defined as an unpleasant experience with sensorial, emotional and cognitive dimensions associated with actual or potential injury.

The detection of noxious stimuli by the neurons is denominated nociception and the neurons that are sensitive to these stimuli are defined as nociceptors. These nociceptors are not usually activated by non-noxious stimuli, as they present a high activation threshold. However, their sensitivity may be increased by inflammation. The cell bodies of the nociceptors are localized in the dorsal root or trigeminal ganglia, according to the region they innervate. These nociceptors make synapse with neurons in the spinal cord dorsal horn or in the brain stem. These secondary neurons project to some structures in the diencephalon, where they make synapse with neurons that project to the cerebral cortex [Woolf, C. J. & Salter, M. W. Science, 288: 1765, 2000].

The sensitization of the nociceptors may result in allodynia and hyperalgesia in the site of the injury or adjacent tissues. The pain may also be reported spontaneously without the need of additional stimuli [Woolf, C. J. & Salter, M. W. Science, 288, 1765, 2000]. The IASP defines hyperalgesia as an exacerbated response to a noxious stimulus and allodynia as pain associated with an innocuous stimulus. These responses are protective mechanisms, as they contribute to behaviour aiming to additional stimulation of the injured site and also to the healing process. The increased responsiveness of the dorsal horn neurons after intense and continuous activation of the nociceptors induces changes of the processing of low and high sensorial stimuli by the central nervous system. Thus, innocuous mechanical stimuli may be interpreted as noxious and may increase the magnitude of pain induced by noxious stimuli [Cervero, F. & Laird, J. M. Pain, 68, 13, 1996]. Some mechanisms involved in the increase of neuronal sensitivity have been identified: increased expression of sodium channels, increased activity of glutamatergic receptors, changes in the effect of gamma-aminobutyric (GABA) on the neuronal excitability and increased calcium influx [Jensen, T. S. Cephalalgia, 21, 765, 2001].

Many of the neuronal changes involved in the pain processing and other manifestations of the inflammatory response may result from the action of a specific group of mediators, the prostaglandins. After the tissue injury, there is a quick induction of the cyclooxygenase (COX) enzyme and the concentrations of eicosanoids, mainly prostaglandins (PG), in the inflammatory exudates are increased. The nonsteroidal anti-inflammatory drugs (NSAIDs) inhibit COX activity and thus the conversion of arachdonic acid to PGG₂ and PGH₂. PGH₂ is substrate to other enzymes that catalyze its conversion to other eicosanoids such as PGD₂, PGE₂, PGI₂, PGF_{2α} and TXA₂ [Bertolini, A.; Ottani, A.; Sandrini, M. Pharmacol. Res., 44, 37, 2001].

Although pain represents the inflammatory symptom most investigated, as it represents the symptom that causes the greatest discomfort the patients, other local manifestations are also associated with the inflammatory response. Among the most important and easily identified, edema and cell migration can be mentioned. The inflammatory edema results from plasma leakage due to vasodilation and increased vascular permeability. The vasodilation associated with blood stasis and increased production of chemotactic factors also contribute to the increased cell migration to the inflammatory site. Among the migrating cells, neutrophils represent the most numerous and play a more important role in the defence response. When the inflammatory response lasts some days or longer, an angiogenic and proliferative response may occur leading to the formation of fibrovascular tissue [Tracey K. J., Nature, 420, 853, 2002].

In addition to local manifestations, some systemic responses may occur. One important component of the inflammatory response, mainly when there is an association with infection and cancer, is fever. Fever corresponds to an increase in internal body temperature above normal amplitude of daily variation and is recognized as a defense mechanism to a pathological process. Certainly, it is the most ancient symptom used as an indication of an infectious status. Fever is caused by an upward change of the hypothalamic set point due to abnormalities in the brain itself or the action of endogenous pyrogens produced by multiple inflammatory and non-inflammatory cells [Kluger, M. J. Physiol. Rev. 71, 93, 1991].

Primarily, the acute inflammatory response represents a defense mechanism, allowing the host to remove cell debris and microorganisms and promote tissue regeneration. However, in some cases the pain and edema associated with acute inflammation may be intense and represent a great discomfort to the patient. Even worse are the cases when the inflammation lasts days or months, resulting in extensive tissue destruction and increased damage to the host. Some examples include rheumatoid arthritis, lupus, psoriasis etc. When the inflammation results in intense pain or edema or when the inflammation becomes chronic, the pharmacotherapy with anti-inflammatory drugs is usually warranted.

The non-steroidal anti-inflammatory drugs (NSAIDs) represent the pharmacologic group most used to attenuate signs and symptoms of inflammation. The NSAIDs represent a group of different drugs that share some common mechanisms of action. Their analgesic and anti-edematogenic effect result from the inhibition of the synthesis of important inflammatory mediators. Among the NSAIDs there are non-selective (diclofenac, indomethacin and ibuprofen) and COX₂ selective inhibitors (celecoxib, rofecoxib and etoricoxib).

Steroid anti-inflammatory drugs, on the other hand, display a wider inhibitory effect on the production of inflammatory mediators. In addition to inhibiting the production of many eicosanoids, they also inhibit the production of inflammatory cytokines, nitric oxide, adhesion molecules, etc. These drugs present potent anti-inflammatory and immunosuppressive activities, justifying their use in the treatment of severe inflammatory conditions such as rheumatoid arthritis, lupus, psoriasis, asthma and anaphylactic shock. Among the most frequent used steroid anti-inflammatory drugs are dexamethasone, prednisone, betamethasone, budesonide and beclomethasone.

Some less conventional drugs have also been used to relieve pain associated with different inflammatory or non-inflammatory conditions. α₂-adrenergic agonists, originally approved as anti-hypertensive drugs, have been used to facilitate the anesthesia, as they present anxiolytic and analgesic activities. They have also been used to alleviate the pain associated with different pathologic conditions when conventional drugs fail [Quan, D. B.; Wandres, D. L.; Schroeder, D. J. Ann. Pharmacother. 27, 313, 1993].

A type of pain, whose relief is not easily attained with the conventional drugs, is that associated with lesions of the brain, spinal cord or peripheral nerves. The neuropathic pain, as it is defined, may occur associated with different forms of cancer, diabetes, amputations, traumatic lesions of nerves, etc. Treatments to reduce the neuronal hyperactivity that characterizes these painful conditions usually provide some relief. Many antiepileptic drugs have been used. Carbamazepine and phenyloin were the first antiepileptics used in the treatment of trigeminal neuralgia, one of the most frequent types of neuropathic pain. Today, other types of neuropathic pain have been shown to be alleviated by antiepileptics and the number of these drugs that also present analgesic activity has been increased. Clinical studies have shown the analgesic efficacy of lamotrigine, gabapentine, pregabaline and topiramate. Valproic acid, thiagabine and felbamate have also been under clinical investigation. The reduction of the neuronal excitability after treatment with these drugs have been attributed to blockade of sodium channels, but other effects may also contribute to their analgesic effect [Jensen, T. S. Cephalalgia 21: 765, 2001].

Although there are different classes of drugs with analgesic activity, there are many painful conditions that are not effectively alleviated by the available drugs.

No pharmaceutical compositions of semicarbazones, thiosemicarbazones and/or their derivatives were found in the State-of-the-Art with analgesic, antipyretic and anti-inflammatory activities, characteristics of the present invention.

### DETAILED DESCRIPTION OF INVENTION

The present invention is characterized by the pharmaceutical compositions of semicarbazone and/or thiosemicarbazone, or a pharmaceutically acceptable salt, hydrated or solvated thereof, mixed with or included into pharmaceutically acceptable excipients, in solution or in the solid state, to be used in the treatment of inflammatory, painful or febrile conditions and also to prevent signs or symptoms of inflammation.

The compounds of the present invention are useful in pharmaceutical compositions with conventional carriers or vehicles, preferentially for oral administration to humans or animals in dosages as tablets, capsules, pills, powders and granules and the necessary quantity of the semicarbazones and/or thiosemicarbazones. In addition, they may be used as suppositories, sterile parenteral solutions, sterile parenteral suspensions, sterile non parenteral solutions or sterile non parenteral suspensions, oral solutions or oral suspensions oil-water or water-oil suspensions, emulsions.

The present invention claims that semicarbazones and thiosemicarbazones, benzaldehyde semicarbazone (BS) as a non-limiting example, present an anti-inflammatory activity. This activity was observed after oral administration of a suspension of BS in carboxymethylcellulose, a pharmaceutical acceptable excipient, and also after parenteral (intraperitoneal) administration of BS dissolved in dimethylsulphoxide. The carboxymethylcellulose is preferably used in concentration range of 0.1 to 5% in compositions for oral administration. The antiinflammatory activity is characterized by inhibition of the nociceptive response induced by formaldehyde, thermal and mechanical allodynia, paw edema and cell migration induced by carrageenan, febrile response induced by bacterial endotoxin and formation of fibrovascular tissue induced by cotton. In addition, acute and sub-acute toxicity test did not indicate that oral administration of BS induce signs or symptoms of toxicity in experimental animals.

Oral or parenteral (intraperitoneal) administration of BS markedly inhibited the nociceptive response induced by formaldehyde in mice. This response involves two distinct phases. The early phase, which starts immediately after the injection and lasts about 5 min, seems to be caused predominantly by direct C-fibre activation. The late phase that starts approximately 15 min after formaldehyde injection and lasts about 15 min seems to depend on the local inflammation and also activation of N-methyl-d-aspartate (NMDA) and non-NMDA receptors and nitric oxide (NO) production in the spinal cord Usually, the second phase of the nociceptive response induced by formaldehyde is inhibited by anti-inflammatory drugs, while both the first and second phases are inhibited by centrally acting drugs such as opioids and antidepressants [Tjolsen et al., Pain 51, 5, 1992]. A more marked inhibition of the second phase of the nociceptive response induced by formaldehyde indicates that BS presents a pharmacological activity that resembles more that of anti-inflammatory drugs. Interestingly, other drugs with anticonvulsant activity such as lamotrigine, carbamazepine and phenyloin also inhibit more effectively the second phase of the nociceptive response induced by formaldehyde [Blackburn-Munro et al., Eur. J. Pharmacol. 445, 231, 2002].

Oral or parenteral (intraperitoneal) administration of BS also inhibited the thermal hyperalgesia and mechanical allodynia induced by carrageenan in rats, but not the nociceptive response of mice in the hot-plate model. The hyperalgesia and allodynia induced by carrageenan are associated with local production of multiple inflammatory mediators that sensitize and activate nociceptors [Handy and Moore, Neuropharmacology, 37, 37, 1998; Poole et al., Br. J. Pharmacol. 126, 649, 1999; Zhang et al., J. Pharmacol. Exp. Ther. 283, 1069, 1997), and with facilitation of the synaptic transmission in the central nervous system resulting from the activation of NMDA [Chapman et al. Br. J. Pharmacol. 116, 268, 1995] and non-NMDA [Sluka et al., Pain 59, 95, 1994] receptors and NO production [Meller et al., Neuroscience 60, 367, 1994]. In this experimental model, drugs that inhibit the production or action of inflammatory mediators such as non-steroidal anti-inflammatory drugs, antagonists of bradykinin receptors and inhibitors of NO synthesis or drugs that inhibit the central nociceptive processing such as inhibitors of NO synthesis and NMDA and non-NMDA antagonists reduce the nociceptive behavior [Poole et al., Br. J. Pharmacol. 126, 649, 1999; Zhang et al., J. Pharmacol. Exp. Ther. 283, 1069, 1997; Chapman et al., Br. J. Pharmacol. 116, 268, 1995; Meller et al., Neuroscience 60, 367, 1994; Sluka et al., Pain 59, 95, 1994]. On the other hand, the nociceptive response in the hot-plate model results from immediate and direct activation of nociceptive afferent fibers by temperatures higher than their activation threshold [Caterina et al., Nature 389, 816, 1997]. An antinociceptive effect in the hot-plate model is usually observed after treatment of the animals with centrally acting drugs such as opioid analgesics [Hammond & Proudfit, Brain Res. 188, 79, 1980], 5-hydroxytryptamine [Ogren & Holm, J. Neural Transm. 47, 253, 1980] and norepinephrine [Tura & Tura, Brain Res. 518, 19, 1990] uptake inhibitors and α2-adrenoceptor agonists [Takano & Yaksh, J. Pharmacol. Exp. Ther. 261, 764, 1992], but not after treatment with drugs that inhibit the synthesis or action of inflammatory mediators [Engelhardt et al., Inflam. Res. 44, 423, 1995, Correa et al., Br. J. Pharmacol. 117, 552, 1996; Fantetti et al., Arzneimittel-Forschung 49, 137, 1999]. The inhibition of carrageenan-induced allodynia but not the nociceptive response in the hot-plate model also gives support for a more marked peripheral action to explain BS antinociceptive effect.

It is unlikely that the oral or parenteral administration of BS induces motor incoordination or a muscle relaxing effect, as the time the mice spent in the rotarod was not changed. Thus, it is unlikely that any effect of BS on the nociceptive response resulted from motor incoordination or muscle relaxing effect. The lack of antinociceptive effect in the hot-plate model also suggests that the BS antinociceptive effect observed in the three previous models is not due to a non-specific effect that could result from motor incoordination or muscle relaxing effect.

Oral or parenteral (intraperitoneal) administration of BS also inhibited carrageenan-induced edema in rats. These results provide further support for an action that resembles more that of anti-inflammatory drugs. The edema induced by carrageenan results from the local action of multiple inflammatory mediators, including prostaglandins bradykinin, NO, 5-hydroxytryptamine and histamine [Wirth et al., Agents and Actions Supplement 38, 428, 1992; Stochla and Maslinski, Agents and Actions 12, 201, 1982; Zhang et al., J. Pharmacol. Exp. Ther. 283, 1069, 1997; Holsapple et al. Agents and Actions 10, 368-373, 1980].

Oral administration of BS also inhibited the proliferative phase of the inflammatory response. In the present invention, it was observed that a seven-day treatment with BS inhibited the formation of fibrovascular tissue induced by a subcutaneous cotton implant in rats. In this model, the cotton induces an inflammatory angiogenic response that reproduces many features of the healing occurring after mechanical and natural injuries such as balloon angioplasty, atherosclerosis, inflamed synovium and surgical wounds. As compounds structurally related to BS act as inhibitors of Na⁺ channels [Ilyin et al., Br. J. Pharmacol. 144, 801, 2005; Shao et al., J. Med. Chem. 47, 4277, 2004], it may be suggested that one probable mechanism by which this drug inhibited the blood vessel formation may be associated with its effect on ion channels. Blockers of Ca²⁺ and Na⁺ channels have been shown to inhibit angiogenesis in experimental models in vivo and in vitro [Alliegro et al., J. Exp. Zool. 267, 245, 1993; Rocha e Silva et al., Inflammation 22, 643, 1998]. In addition, anti-inflammatory drugs, including steroidal and non-steroidal, inhibit angiogenesis in vivo and in vitro [Hori et al., Br. J. Pharmacol 118, 1584, 1996; Jones et al., Nature Med. 5, 1418, 1999; Ghosh et al., J. Pharmacol. Exp. Ther. 295, 8802, 2000]. As BS presents a profile that resembles that of anti-inflammatory drugs, it may inhibit the synthesis or release of inflammatory mediators that contribute to the formation of new blood vessels.

Oral administration of BS also inhibited the cell migration induced by intraperitoneal injection of carrageenan in rats. In this model, carrageenan induces the production of multiple inflammatory mediators in the peritoneal cavity. These mediators stimulate cell chemotaxis, predominantly neutrophils. It is possible that BS inhibits the production or action of inflammatory mediators that contributes to cell migration.

Finally, oral administration of BS inhibited the febrile response induced by intravenous injection of bacterial endotoxin in rats. It has been shown that endotoxin stimulates the production of many endogenous pyrogens, including interleukin-1, interleukin-6, interferons, tumor necrosis factor and prostaglandins, that changes the activity of hypothalamic neurons. Such changes result in an increase of heat production and reduction of heat loss and, consequently, the increase of body temperature [Kluger, M. J. Physiol. Rev. 71, 93, 1991]. As BS presents a profile that resembles more that of anti-inflammatory drugs, its antipyretic activity may be associated with the inhibition of production of pyrogenic mediators. Importantly, BS per se did not induce changes of body temperature, indicating that the effect observed in the pyrogenic test represents a true antipyretic effect.

This invention can be better understood by use of some non-limiting examples, as follows:

### Example 1

### Assessing the Effect of Semicarbazones, Thiosemicarbazones and Combinations on Motor Activities of Mice, Using Benzaldehyde Semicarbazone as a Non-Limiting Example

The effect of BS on motor activity of Swiss male mice (20-30 g) was evaluated in order to investigate whether inhibition of nociceptive behavior in animals treated with BS would be a result of a central depressive effect. Their motor activity was evaluated in a rotarod. A day before the experiment, the mice were trained in the apparatus. During the experiment, they were put in a rotarod (14 rpm) and the time they stayed in the apparatus was determined. One minute was the cut-off time. After basal measurements, the animals were treated with BS (10, 25 or 50 mg/kg, intraperitoneal or 100 or 200 mg/kg, oral). Dimethyl sulfoxide (DMSO) 25%+Tween 80 10% in saline was the vehicle used for intraperitoneal administration and carboxymethylcellulose 0.5%, a pharmaceutically acceptable excipient, was used for oral administration. Thirty or 60 min after intraperitoneal or oral administration, respectively, the animals were evaluated again in the rotarod apparatus. The results showed that BS did not alter the motor activity of the animals. This is an important result when evaluating results in the pain models as they suggest that any effect in such models does not result from motor incoordination or muscle relaxing effect induced by BS.

### Example 2

### Assessing the Effect of Semicarbazones, Thiosemicarbazones and Combinations on the Nociceptive Response Induced by Formaldehyde in Mice, Using Benzaldehyde Semicarbazone as a Non-Limiting Example

In the nociceptive response induced by formaldehyde (0.92%, 20 μL), the inflammatory stimulus is injected subcutaneously into the dorsum of the right hind paw of male Swiss mice. The time the animal spent licking its injected paw was determined from 0 to 5 min (first phase) and from 15 to 30 min (second phase) after formaldehyde injection.

Dimethyl sulfoxide (DMSO) 25%+Tween 80 10% in saline was the vehicle used for intraperitoneal administration and carboxymethylcellulose 0.5%, a pharmaceutically acceptable excipient, was used for oral administration. Thirty or 60 min after intraperitoneal or oral administration, respectively, formaldehyde was injected and the nociceptive behavior evaluated as described.

Only the dose of 50 mg/kg of BS, administered via the intraperitoneal route, inhibited the first phase of the nociceptive response induced by formaldehyde. However, the second phase of the nociceptive response induced by formaldehyde was markedly inhibited by previous intraperitoneal or oral administration of BS (10, 25 or 50 mg/kg, i.p. or 100 or 200 mg/kg, oral).

### Example 3

### Assessing the Effect of Semicarbazones, Thiosemicarbazones and Combinations on Thermal and Mechanical Allodynia Induced by Carrageenan in Rats, Using Benzaldehyde Semicarbazone as a Non-Limiting Example

BS has also inhibited the nociceptive response in the model of thermal and mechanical allodynia induced by carrageenan in Wistar male rats (200-250 g). In the model of thermal allodynia, the latency for paw removal after application of a thermal stimulus is assessed in the Hargreaves apparatus (model 7370, Ugo Basile, Italy). In the model of mechanical allodynia, the frequency of paw withdrawal to a series of ten touches with a nylon filament to the plantar surface of the right hind paw is determined.

Dimethyl sulfoxide (DMSO) 25%+Tween 80 10% in saline was the vehicle used for intraperitoneal administration and carboxymethylcellulose 0.5%, a pharmaceutically acceptable excipient, was used for oral administration. Thirty or 60 min after intraperitoneal or oral administration, respectively, carrageenan (1%, 50 μl, suspended in saline) was injected into the plantar surface of the right hindpaw and the latency for paw removal after application of a thermal stimulus or the frequency of paw withdrawal to the mechanical stimulus was evaluated as described.

The previous treatment of the animals with BS (50 mg/kg, i.p., −30 min; 100 or 200 mg/kg, oral, −60 min) inhibited both thermal and mechanical allodynia induced by carrageenan.

### Example 4

### Assessing the Effect of Semicarbazones, Thiosemicarbazones and Combinations on Paw Edema Induced by Carrageenan in Rats, Using Benzaldehyde Semicarbazone as a Non-Limiting Example

The paw edema was assessed using a pletismometer (model 7140, Ugo Basile, Italy). The basal paw volume was determined before any treatment and the edema was induced by intraplantar injection of carrageenan into the right hindpaw (1%, 50 μl, suspended in saline).

Dimethyl sulfoxide (DMSO) 25%+Tween 80 10% in saline was the vehicle used for intraperitoneal administration and carboxymethylcellulose 0.5%, a pharmaceutically acceptable excipient, was used for oral administration. Thirty or 60 min after intraperitoneal or oral administration, respectively, carrageenan (1%, 50 μl, suspended in saline) was injected and the paw edema was evaluated as described.

BS (50 mg/kg, i.p., −30 min or 200 mg/kg, oral, −60 min) inhibited the paw edema induced by carrageenan.

### Example 5

### Assessing the Effect of Semicarbazones, Thiosemicarbazones and Combinations on the Nociceptive Response of Mice Induced by Heat in the Hot Plate Model, Using Benzaldehyde Semicarbazone as a Non-Limiting Example

In this model, the mice were exposed to a heat metal surface (54° C.) and the latency for licking the paws or jumping off the plate was determined.

Dimethyl sulfoxide (DMSO) 25%+Tween 80 10% in saline was the vehicle used for intraperitoneal administration and carboxymethylcellulose 0.5%, a pharmaceutically acceptable excipient, was used for oral administration. Thirty or 60 min after intraperitoneal or oral administration, respectively, the mice were exposed to the hot plate and the latency for displaying the nociceptive behavior was determined as described.

BS (10, 25 or 50 mg/kg, i.p., −30 min; 100 or 200 mg/kg, oral, −60 min) did not induce an antinociceptive effect in this model.

### Example 6

### Assessing the Effect of Semicarbazones, Thiosemicarbazones and Combinations on the Fibrovascular Tissue Formation Induced by a Subcutaneous Implant of Cotton in Mice, Using Benzaldehyde Semicarbazone as a Non-Limiting Example

In the model of fibrovascular tissue formation, a cotton pellet (10 mg) is subcutaneously implanted in the back of mice. After seven days, the mice are euthanized and the cotton pellet involved plus the surrounding fibrovascular tissue is removed and its mass determined.

Carboxymethylcellulose 0.5%, a pharmaceutically acceptable excipient, was used for oral administration. BS (200 mg/kg·day, oral) was administered during seven days. Twenty-four hours after the last administration, the animals were euthanised and the mass of the cotton pellet plus the surrounding fibrovascular tissue was determined as described.

BS (200 mg/kg·per day, per os, 7 days) inhibited the fibrovascular tissue formation.

### Example 7

### Assessing the Effect of Semicarbazones, Thiosemicarbazones and Combinations on the Febrile Response Induced by Bacterial Endotoxin in Rats, Using Benzaldehyde Semicarbazone as a Non-Limiting Example

In this model, endotoxin of E. coli (50 μg/kg) is injected intravenously in rats and the colonic temperature is determined during 6 h.

Carboxymethylcellulose 0.5%, a pharmaceutically acceptable excipient, was used for oral administration. BS (200 mg/kg, oral, −60 min) inhibited the febrile response induced by endotoxin. BS (200 mg/kg, oral) did not alter colonic temperature per se.

### Example 8

### Assessing the Effect of Semicarbazones, Thiosemicarbazones and Combinations on the Cell Migration Induced by Intraperitoneal Injection of Carrageenan in Rats, Using Benzaldehyde Semicarbazone as a Non-Limiting Example

In this model, carrageenan (500 μg, 1 ml, suspended in saline) is injected into the peritoneal cavity of rats. After three hours, the animals are euthanized and 10 ml phosphate buffer are injected into the peritoneal cavity. The peritoneal lavage is removed and the centrifuged. The supernatant is removed and the pellet is suspended in a 2 ml of phosphate buffer. An aliquot of 20 μl is added to a 400 μl Turk's solution. The number of cells is determined by microscopy.

Carboxymethylcellulose 0.5%, a pharmaceutically acceptable excipient, was used for oral administration. BS (200 mg/kg, oral, −60 min) inhibited cell migration induced by carrageenan.

### Example 9

### Toxicity Assessment of Semicarbazones and/or Thiosemicarbazones as Well as Their Derivatives and Combinations of the Latter

Toxicological studies were based on established protocols (OECD Test Guidelines) internationally recognized as a reference standard tool for chemical tests.

The OECD 420 guideline—Fixed-Dose Procedure for Assessing Oral Acute Toxicity (OECD, 2001) was followed. Only one dose of substances (300 mg/kg or 2000 mg/kg) was administered orally in rats. A careful observation of the rats was made 30 minutes later and repeated hourly up to 12 h. Food was not provided during the first 4 h and the animals were observed for more than 13 days (daily, from 11:00 am to 1:00 pm). After that, the animals were euthanised and submitted to macroscopic and microscopic necropsy.

The OECD 407 guideline—Repeated-dose Procedure for Assessing Oral Subacute Toxicity (OECD, 1995) was used. The test substance was administered orally daily (from 11:00 am to 1:00 pm) during 28 days. The animals were observed daily during the whole period. Doses of 100, 300, 500 mg/kg for BS were used.

Laboratory analyses for acute (macroscopic and microscopic analyses) and subacute (macroscopic and microscopic analyses, biochemical and hematologic analysis) toxicity tests were carried out.

A death occurrence as a result of administration of the highest dose (2.000 mg/kg) in the acute toxicity test has led to the classification of BS in the category 5 in the Globally Harmonized System for the Classification and Labeling of Chemicals. This means that this compound show low acute toxicological risk, although they can be dangerous for vulnerable population in certain circumstances (United Nations, 2005).

In the acute toxicity test, a dose of 300 mg/kg, induced reduction of motor activity and grooming, reversible after few hours. As this dose did not induce evident toxicity, a 2000 mg/kg dose was then used. This higher dose induced some signs of toxicity, as indicated in Table 1. As to BS, reversibility occurred in the second day.

Table 2 also shows the body weight changes induced by treatment of the rats with BS during 28 days. The higher doses of BS reduced body weight gain.

TABLE 1
Evaluation of acute toxicity of BS and BS-β-CD (single dose).

| | Dose | | Observation | |
|---|---|---|---|---|
| Group | (mg/kg) | T/M | period | Observed signs |
| BS | 0 | 10.0 | 30′ - 14th day | Normal aspects |
| | 300 | 5/0 | 30′ - 9 h | General activity (3), |
| | | | | grooming (1) |
| | | | 9 h - 14th day | No toxicity signs |
| | 2000 | 5/1* | 30′ - 12 h | Ataxia (4), cage corner (1), |
| | | | | piloerection (1), |
| | | | | prostration (3) |
| | | | From the 2^{nd} | No toxicity signs |
| | | | day | |
| BS-β- | 0 | 10.0 | 30′ - 14th day | Normal aspects |
| CD | 300 | 5/0 | 30′ - 12 h | General activity (2), |
| | | | | grooming (2) |
| | | | From the 2^{nd} | No toxicity signs |
| | | | day | |
| | 2000 | 5/1 | 30′ - 12 h | Ataxia (4), general |
| | | | | activity (1), cage corner |
| | | | | (1), spasm (1), grip strength |
| | | | | (1), grooming (1), piloerection |
| | | | | (1), prostration, (1) |
| | | | From the 3^{rd} | No toxicity signs |
| | | | day | |

T/M = number of rats treated/number of deaths.
Observations after administration = 30 min, 1 h and at every 1 h until the 12^{th} hour at every 24 h (two times a day) until the 14^{th} day.
Normal score of signs observed: ataxia (0), general activity (4), cage corner (0), spasm (0), grip strength (4), grooming (0), piloerection (0), prostration (0), reflex (4), bodily tonus (4).
*animal euthanised.

TABLE 2
Evaluation of body weight changes induced by BS and BS-β-CD.

| | BS | | | BS-β-CD | | |
|---|---|---|---|---|---|---|
| | | Initial body | Final body | | Initial body | Final |
| Sex | Dose | mass | mass | Dose | mass | body mass |
| Males | 0 | 234.90 ± 22.04^{A} | 344.10 ± 32.05^{A} | 0 | 231.00 ± 3.52^{A} | 299.50 ± 14.02^{A} |
| | 100 | 240.87 ± 20.07^{A} | 340.20 ± 50.91^{A} | 25 | 231.80 ± 8.65^{A} | 281.40 ± 20.72^{A} |
| | 300 | 192.50 ± 8.32^{A} | 258.53 ± 25.76^{B} | 75 | 239.20 ± 14.09^{A} | 275.70 ± 22.68^{A} |
| | 500 | 225.30 ± 34.72^{A} | 240.00 ± 46.65^{B} | 125 | 236.80 ± 12.94^{A} | 297.70 ± 8.68^{A} |
| | Satellite | 197.00 ± 4.51^{A} | 222.57 ± 29.34^{B}* | Satellite | 239.20 ± 12.76^{A} | 293.70 ± 15.42^{A} |
| Females | 0 | 210.00 ± 7.81^{A} | 225.80 ± 10.65^{A} | 0 | 169.60 ± 5.98^{A} | 179.70 ± 8.11^{C} |
| | 100 | 207.60 ± 12.62^{A} | 230.30 ± 15.42^{A} | 25 | 162.80 ± 14.53^{A} | 192.00 ± 18.91^{BC} |
| | 300 | 192.30 ± 13.34^{A} | 208.38 ± 24.28^{AB} | 75 | 170.00 ± 7.17^{A} | 197.90 ± 21.99^{AB} |
| | 500 | 196.60 ± 8.20^{A} | 170.00 ± 17.22^{B} | 125 | 166.10 ± 7.42^{A} | 213.10 ± 6.38^{A} |
| | Satellite | 198.10 ± 15.06^{A} | 197.27 ± 14.44^{AB} | Satellite | 169.90 ± 9.10^{A} | 212.80 ± 17.81^{A} |

Data are expressed with as mean ± sem (n = 5).
Letters refer to comparison by sex, by drug for each period. Duncan test (P < 0.05).
*Death of an animal during treatment.

1. A pharmaceutical composition comprising at least a semicarbazone or a thiosemicarbazone, or a pharmaceutically acceptable salt, hydrated or solvated thereof, according to the structural formula 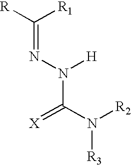
in which R, R1, R2 and R3 are H, aryl or alkyl groups and X is oxygen or sulfur, for the treatment of inflammatory, febrile and painful inflammatory conditions, inflammatory edema and peripheral or central neurophatic painful conditions or prevention of signs and symptoms of inflammation.

2. The pharmaceutical composition of claim 1 comprising a therapeutically effective amount of at least a semicarbazone, or a thiosemicarbazone, or a pharmaceutically acceptable salt, hydrated or solvated thereof, mixed or included in a pharmaceutically acceptable carrier or excipient.

3. The pharmaceutical composition of claim 1, wherein said a semicarbazone or a thiosemicarbazone is provided as sustained or controlled release systems for human and veterinary use.

4. The pharmaceutical composition of claim 2, wherein the pharmaceutical excipient is carboxymethylcellulose, in a concentration range from 0.1 to 5%, for oral administration.

5. The pharmaceutical composition of claim 2, wherein the pharmaceutical excipient is starch, lactose, microcrystalline cellulose, hydroxypropylmethylcellulose, talc, magnesium stearate or a mixture thereof and the composition is in the solid form for oral administration.

6. The pharmaceutical composition claim 2, wherein the pharmaceutical excipient is propyleneglycol, glycerol, sorbitol, saccharose, glucose or fructose and the composition is in the liquid form for oral administration.

7. The pharmaceutical composition of claim 1, wherein the pharmaceutical excipient is polyvinyl pyrrolidone, cremophor, tween 80 the composition is in the liquid form for parenteral administration.

8. A method of treating inflammatory, painful inflammatory conditions, inflammatory edema, and peripheral painful conditions comprising administering to a subject in need of such treatment a pharmaceutically effective amount of a compound as defined in claim 1.

9. The method of claim 8 wherein the subject is a human.

10. The method of claim 8 wherein the subject is an animal.

11. A method of treating febrile conditions comprising administering to a subject in need of such treatment a pharmaceutically effective amount of a compound as defined in claim 1.

12. The method of claim 11 wherein the subject is a human.

13. The method of claim 11 wherein the subject is an animal.

14. A method of treating peripheral or central neurophatic painful conditions comprising administering to a subject in need of such treatment an effective amount of a compound as defined in claim 1.

15. The method of claim 14 wherein the subject is a human.

16. The method of claim 14 wherein the subject is an animal.

17. A method of preventing signs and symptoms of inflammation comprising administering to a subject in need of such treatment an effective amount of a compound as defined in claim 1.

18. The method of claim 17 wherein the subject is a human.

19. The method of claim 17 wherein the subject is an animal.
